(19) United States
(12) Patent Application Publication
(10) Pub. No.: US 2012/0301897 A1
(43) Pub. Date: Nov. 29, 2012

(54) ACETAMINOPHEN-PROTEIN ADDUCT ASSAY DEVICE AND METHOD

(75) Inventors: Laura James, Little Rock, AR (US); Dean Roberts, Little Rock, AR (US); Jack A. Hinson, Little Rock, AR (US)

(73) Assignee: BOARD OF TRUSTEES OF THE UNIVERSITY OF ARKANSAS, Little Rock, AR (US)

(21) Appl. No.: 13/566,516

(22) Filed: Aug. 3, 2012

Related U.S. Application Data
(62) Division of application No. 12/427,434, filed on Apr. 21, 2009.
(60) Provisional application No. 61/046,673, filed on Apr. 21, 2008.

(51) Int. Cl. G01N 33/558; G01N 21/64; G01N 21/78; G01N 33/53

(52) U.S. Cl. 435/7.9; 436/501

(57) ABSTRACT

The present invention describes devices and methods for detecting and measuring the amount of acetaminophen-protein adducts in a sample.

### CROSS REFERENCE TO RELATED APPLICATION

This application is a divisional of U.S. patent application Ser. No. 12/427,434, filed Apr. 21, 2009, which claims priority from U.S. provisional patent application Ser. No. 61/046,673, entitled “Acetaminophen-Protein Adduct Chromatographic Assay Device and Method” filed on Apr. 21, 2008, both of which are hereby incorporated by reference in their entirety.

### FIELD OF THE INVENTION

The present invention relates to devices and methods for detecting and measuring the amount of acetaminophen-protein adducts in a sample.

### BACKGROUND OF THE INVENTION

Acetaminophen (APAP) is the most common pharmaceutical product associated with drug toxicity. In severe cases, APAP overdose may lead to acute liver failure (ALF) and death. Over 100,000 telephone calls concerning APAP overdose are made to poison control centers in the U.S. annually. The FDA estimates that approximately 450 deaths are related to APAP overdose annually. For patients that seek treatment within 24 hours of an APAP overdose, and are able to provide accurate information regarding the time and amount of APAP ingested, APAP overdose is relatively straightforward to diagnose and treat.

The diagnosis of APAP overdose is typically based on a determination of an elevated APAP level in peripheral blood. Treatment decisions are based on a comparison of the patient's APAP level to a toxic APAP threshold determined from the time lapsed since the overdose, commonly referred to as the Rumack nomogram. However, the Rumack nomogram as a diagnostic instrument may not be very useful in patients with confounding factors such as presentation to the hospital 24 hours after the overdose, ethanol use, chronic supratherapeutic ingestions, or the use of sustained release APAP formulations. Further, elevated bilirubin levels may interfere with the accuracy of APAP concentration determinations. In patients whose time of ingestion is unknown or patients with chronic toxic exposures, elevated APAP levels are of limited value as a diagnostic tool.

Other laboratory tests may also be used to help determine the presence and severity of APAP overdose. Some lab tests, such as serum alanine aminotransferase (ALT) and serum aspartate aminotransferase (AST) indicate the occurrence of liver damage, but neither bioindicator is specific to APAP overdose.

Acetaminophen toxicity is mediated by conversion of acetaminophen to a reactive metabolite, N-acetyl-p-benzoquinone imine (NAPQI). NAPQI covalently binds to cysteine groups in proteins or peptides to form APAP-protein adducts mainly in the liver and to a lesser degree in other tissues capable of metabolizing APAP. The APAP-protein adducts appear in the serum, tissues, and other body fluids due to cell toxicity and associated cellular membrane lysis and are a specific biomarker of acetaminophen toxicity. These APAP-protein adducts have the cysteine sulfur group covalently attached to the APAP ring meta to the acetamido group and ortho to the phenol group, and are also called 3-(cystein-S-yl) APAP (3-Cys-A)-protein adducts.

Antibodies with specificity for APAP-protein adducts have been developed and laboratory tests based on these antibodies (enzyme linked immunosorbent assays, Western blots, and immunohistochemical determination of APAP-protein adducts) have been used to detect the presence of APAP-protein adducts and assess APAP toxicity. APAP-protein adducts have also been detected by high performance liquid chromatography with electrochemical detection (HPLC-EC). The existing antibody assays and the HPLC-EC methodology are research laboratory based, require sophisticated laboratory equipment, trained laboratory technicians, and several hours or more to obtain results.

A need exists in the art for an assay that yields measurements that may be used to diagnose an APAP overdose rapidly, specifically, and accurately. In addition, the assay should use relatively simple equipment and methods, so that the diagnostic measurements may be rapidly obtained and analyzed by caregivers with little specialized training in laboratory techniques.

### SUMMARY OF THE INVENTION

The present invention provides a device for use in conducting a competitive assay that detects an amount of APAP-protein adduct in a sample. The device includes an amount of anti-APAP antibody coupled to an indicator and an amount of synthetic APAP-protein adduct. In another aspect, the device includes an amount of synthetic APAP-protein adduct coupled to an indicator and an amount of anti-APAP antibody. In yet another aspect, the device for use in conducting a sandwich assay includes an amount of a first anti-APAP antibody coupled to an indicator and an amount of a second anti-APAP antibody. In still another aspect, the device includes an anti-APAP antibody.

Another aspect provides a dipstick device for detecting and quantifying an APAP-protein adduct in a sample. The dipstick device includes an amount of a synthetic APAP-protein adduct coupled to a nanoparticulate gold indicator and an amount of an anti-APAP antibody. The synthetic APAP-protein adduct is diffusively attached at the sample contact end of a substrate. In addition, the synthetic APAP-protein adduct may be APAP bound to BSA, APAP bound to ovalbumin, APAP bound to lactalbumin and combinations thereof. The anti-APAP antibody is adhered to the substrate in a test zone. The antigenic determinant recognized by the anti-APAP antibody is APAP conjugated to a protein containing a free cysteinyl sulfhydryl group.

In another aspect, the synthetic APAP-protein adduct is bound to the substrate, and the anti-APAP antibody is coupled to a nanoparticulate gold indicator. In this aspect, the anti-APAP antibody is diffusively bound to the sample contact end of the substrate.

Yet another aspect provides a dipstick device for detecting and quantifying an APAP-protein adduct in a sample that includes an amount of a first antibody coupled to a nanoparticulate gold indicator and an amount of a second antibody adhered to a substrate in a test zone. The first antibody is diffusively attached at the sample contact end of the substrate. The antigenic determinant recognized by both the first anti-APAP antibody and the second anti-APAP antibody is selected from the group consisting of a 3-(cysteine-S-yl) linkage of the APAP-protein adduct, an exposed portion of the APAP in the APAP-protein adduct, and an APAP hapten.

Still another aspect provides a method of determining an amount of APAP-protein adduct in a sample. The method includes contacting an amount of the sample with a substrate that includes an amount of anti-APAP antibody coupled to an indicator and a synthetic APAP-protein adduct. The method also includes determining the amount of the APAP-protein adduct in the sample by measuring an indicator change caused by the binding of the anti-APAP antibody coupled to indicator with the synthetic APAP-protein adduct.

In another aspect, the method includes contacting an amount of the sample with a substrate comprising an amount of a synthetic APAP-protein adduct coupled to an indicator and an anti-APAP antibody. The amount of APAP-protein adduct in a sample is determined by measuring an indicator change caused by the binding of the synthetic APAP-protein adduct to the anti-APAP antibody.

In yet another aspect, the method includes contacting an amount of the sample with a substrate comprising an amount of a first anti-APAP antibody conjugated with an indicator and a second anti-APAP antibody. The amount of APAP-protein adduct is determined by measuring an indicator change caused by the binding of the synthetic APAP-protein adduct to the anti-APAP antibody.

Other aspects and iterations of the invention are described more thoroughly below.

### BRIEF DESCRIPTION OF THE DRAWINGS

FIG. 1 depicts a competitive assay design of the chromatographic device of the present invention.

FIG. 2 depicts a competitive assay design of the chromatographic device of the present invention.

FIG. 3 depicts a non-competitive assay design of the chromatographic device of the present invention.

FIG. 4 depicts a chromatographic device as the sample containing analyte reaches the test zone of the device.

FIG. 5 depicts a chromatographic device as the sample containing analyte reaches the control zone of the device.

FIG. 6 depicts a non-competitive chromatographic device as the sample containing analyte reaches the test zone of the device.

FIG. 7 depicts the mean concentrations of APAP-protein adducts as a function of time after APAP overdose.

FIG. 8 depicts the measured serum APAP-protein adduct concentrations plotted as a function of the measured serum aspartate aminotransferase (AST) concentration.

FIG. 9 depicts a summary of the mean serum APAP-protein adduct concentrations grouped by severity of toxicity as indicated by AST level.

### DETAILED DESCRIPTION OF THE INVENTION

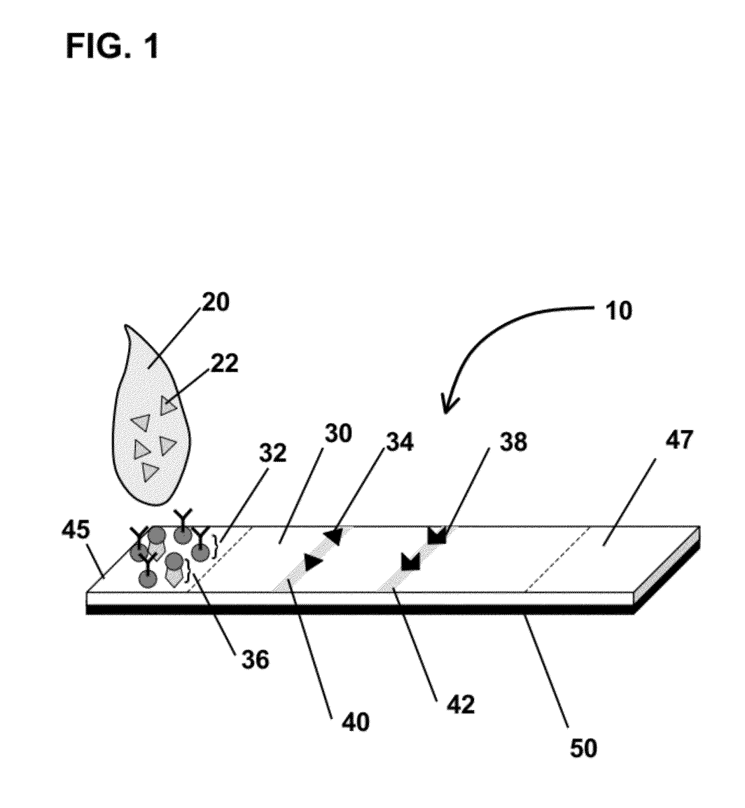

The present invention relates to devices and methods that use anti-APAP antibodies to detect the presence of APAP-protein adducts. More specifically, the present invention provides an APAP-protein adduct detection device, hereinafter referred to as the device 10. An embodiment of device 10 is illustrated in FIG. 1. The device 10 detects an amount of APAP-protein adduct 22 in a sample 20. The device 10 includes an antibody that binds to APAP-protein adduct 22 at an exposed antigenic domain present on APAP bound to cysteine residues of adduct proteins. This antibody could be an antibody with specificity for APAP-protein adduct, or an antibody with specificity for the parent (APAP) drug as either would recognize and bind to APAP-protein adducts, and is hereafter referred to as anti-APAP antibody or anti-APAP (protein) adduct antibody. The device 10, in one embodiment shown in FIG. 1, includes a substrate 30, an amount of anti-APAP antibody coupled to indicator 32, and an amount of synthetic APAP-protein adduct 34. The present invention further provides methods of estimating an amount of APAP-protein adduct 22 in a sample 20.

### I. APAP-Protein Adduct Detection Device

Various embodiments of the device 10 include chromatographic devices and electrochemical devices.

### A. Chromatographic Device

One embodiment is a device 10 used to detect an amount of APAP-protein adduct 22 using a competitive or sandwich assay that incorporates a visual indicator. Generally, the chromatographic device 10 indicates the presence and amount of APAP-protein adduct 22 in a sample 20 by a change in the visual indicator at the test zone 40 of the substrate 30. The indicator change may be in the visible spectrum, the infrared spectrum, or the ultraviolet spectrum. The indicator change may be visible to the unaided eye, or the indicator change may be detected and/or quantified by instruments such as densitometers. In other embodiments, the indicator changes occur in response to the presence of APAP-protein adduct 22 in the sample 20. In still other embodiments, the indicator changes occur in response to the absence of APAP-protein adduct 22 in the sample 20. In an exemplary embodiment, the device 10 is a lateral flow immunochromatographic assay device 10 that includes an anti-APAP antibody coupled to indicator 32. In another exemplary embodiment, the device 10 is a dipstick device.

### B. Electrochemical Device

One embodiment of the invention is a device 10 to detect an amount of APAP-protein adduct 22 using electrochemical methods to detect the reaction of anti-APAP antibodies with synthetic or sample APAP-protein adducts. Generally, an electrochemical device 10 (not shown) indicates the presence and amount of APAP-protein adduct 22 in a sample 20 by changes in the electrical properties of the device 10, including conductivity, resistance, current, electric potential, and combinations thereof. In one embodiment, the device 10 includes anti-APAP antibodies 31 that bind to the APAP-protein adduct 22. Either the anti-APAP antibodies 31 or the synthetic APAP-protein adduct 34 may be immobilized to electrodes. The anti-APAP antibodies 31 or the synthetic APAP-protein adduct 34 may also be suspended in an electrolytic solution using known methods. When APAP-protein adduct 22 binds to the anti-APAP antibodies 31, the electrochemical properties of the device 10 may change and be detected by means of known methods such as a change in the activation of attached redox moieties, the activation of attached conductive moieties, the completion of electrical connections between electrodes by APAP-protein adduct-antibody complexes, interaction of the APAP-protein adduct-antibody complexes with electrochemical signal molecules, or the displacement of competing redox or conductive moieties from the antibodies. The electrochemical device (not shown) may obtain measurements through known electrochemical methods such as direct reduction or oxidation, adsorption stripping voltammetry, cyclic voltammetry, coulometry, chronocoulometry, and combinations thereof.

### II. Sample

Samples 20 that are suitable for use with the chromatographic device 10 of the present invention are any fluid samples or tissue extracts collected from living or post-mortem humans, mice, rats, rabbits, cats, dogs, horses, cows, pigs, or other mammals, selected from the list including blood, urine, saliva, tears, breast milk, lymph, blood plasma, blood serum, bile fluid, cerebrospinal fluid, supernate from cell cultures, tissue extracts, and combinations thereof.

Prior to analyzing the samples 20 using the chromatographic device 10, the samples 20 may be preconditioned using known methods including dilution, protein precipitation, centrifugation, fast dialysis, gel filtration to remove small molecular weight compounds of less than 5 kDa, and combinations thereof.

### III. APAP-Protein Adducts

The APAP-protein adduct 22 detected by the device 10 include acetaminophen covalently bound to a cysteine residue of a protein. In one embodiment, the APAP-protein adduct 22 that is targeted for detection by the device 10 are 3-(cystein-S-yl) APAP (3-Cys-A)-protein adducts. In this embodiment, the anti-APAP antibody 31 used to detect APAP-protein adducts may have specificity for 3-(cystein-S-yl) APAP (3-Cys-A)-protein adducts or have specificity for the parent drug acetaminophen but also recognize acetaminophen bound to protein as 3-(cystein-S-yl) APAP (3-Cys-A)-protein adducts. In an exemplary embodiment, the APAP-protein adduct 22 is acetaminophen covalently bound to albumin at one of the cysteine residues of the albumin.

### IV. Substrate

The substrate 30 of the chromatographic device 10 provides a matrix for conducting an assay. Specifically, the substrate 30 can filter, absorb and transport the sample 20, along with any dissolved APAP-protein adduct 22 and mobile reagents such as anti-APAP antibody coupled to indicator 32. In some embodiments of the device 10 such as a dipstick, the substrate 30 also functions to immobilize other reagents, such as synthetic APAP-protein adduct 34, in a specified area of the substrate 30. The substrate 30 of the chromatographic device 10 may be any porous material such as nitrocellulose, cellulose, paper, glass fiber mesh, silica gel, synthetic resins, and combinations thereof. The substrate 30 may also include a filter membrane 44 (not shown) on the surface or on the sample contact end 45 of the substrate 30 through which the sample 20 is contacted with the substrate 30. The filter membrane 44 functions to prevent blood cells and other undesired particulate matter from entering the substrate 30. The filter membrane 44 is selected to have an average pore size ranging between about 50 μm and about 0.1 mm, and is fabricated from a material selected from the list including polypropylene, PE (polyethylene), PTFE (polytetrafluoroethylene) or other synthetic polymers, glass, metal, and combinations thereof.

The substrate 30 material is selected to be capable of absorbing and transporting the sample 20 using a capillary transport mechanism such as wicking. In particular, the pore size must allow capillary diffusion of large proteins and complexes such as APAP-protein adduct 22 bound to antibody coupled to indicator 32. The substrate 30 material is also selected to maintain its function and structural integrity during storage and subsequent use. To this end, the substrate 30 may be adhesively mounted on a reinforcing backing 50 made from a thin layer of a non-reactive and water-resistant material including glass, a medical grade plastic, a synthetic polymer such as polyethylene, polyurethane, and polypropylene, and combinations thereof.

### A. Test Zone

The substrate 30 may include one or more test zones 40. In one embodiment, the substrate 30 includes a test zone 40 in which an immobilized reagent is adhered. Depending on the particular assay utilized by the device, the immobilized reagent may include, but is not limited to one or more anti-APAP antibody 31, and a synthetic APAP-protein adduct 34. In this embodiment, the immobilized reagent may capture motile reagents and/or an APAP-protein adduct 22 that are dissolved by the solvent of the sample 20 as the solvent wicks past the one or more test zones 40. Motile reagents include, but are not limited to, an anti-APAP antibody coupled to indicator 32, a synthetic APAP-protein adduct coupled to an indicator 35, and combinations thereof. During the detection of APAP-protein adduct 22 using the device 10, a high concentration of motile reagent captured by the immobilized reagent results in a high concentration of indicator within the test zone 40, causing an indicator change.

In one embodiment, the test zone 40 is located between the sample contact end 45 and the distal end 47 of the substrate 30. The distance of the test zone 40 from the sample contact end 45 is selected to allow adequate time for any reactions to take place between the motile reagents and the sample 20 prior to reaching the test zone 40. In another embodiment, the test zone 40 is a narrow band across the width of the substrate 30, as shown in FIG. 1. The test zone 40 may be other shapes and sizes including geometric shapes such as a circle, a square, a triangle, and a diamond, alphanumeric characters, and symbols such as a plus sign and a minus sign.

In yet another embodiment (not shown), the substrate 30 may include at least two test zones 40. In this embodiment, each test zone 40 contains a different immobilized reagent. For example, a device 10 may include a test zone 40a containing an immobilized antibody 31a that has a particular protein in an APAP-protein adduct 22 as an antigenic determinant, and a second test zone 40b containing a second immobilized antibody 31b that has a second protein in an APAP-protein adduct as an antigenic determinant.

### B. Control Zone

The substrate 30 may optionally include a control zone 42 in which an immobilized control capture agent 38 is adhered. Like the test zone 40, a high concentration of motile control compound coupled to an indicator 36 captured by the immobilized control capture agent reagent 38 results in a high concentration of indicator within the control zone 42, causing an indicator change.

In one embodiment, the control zone 42 is located between test zone 40 and the distal end 47 of the substrate 30. In another embodiment, the control zone 42 is a narrow band across the width of the substrate 30, as shown in FIG. 1. The control zone 42 may be other shapes and sizes including geometric shapes such as a circle, a square, a triangle, and a diamond, alphanumeric characters, and symbols such as a plus sign and a minus sign.

### V. Anti-APAP Antibody

The anti-APAP antibody 31 binds to any available APAP-protein adduct 22, including APAP-protein adduct 22 present in the sample 20 as well as synthetic APAP-protein adduct 34. In one embodiment, the major antigenic determinant of the anti-APAP antibody 31 may include the cysteinyl sulfhydryl group on a peptide or protein covalently bound ortho to the hydroxyl group and meta to the acetamide on 3-(cystein-S-yl) APAP, regardless of the identity of the protein. In other embodiments, the anti-APAP antibody 31 may bind to the APAP-protein adduct 22 by virtue of specificity or reactivity with antigenic epitopes of the APAP molecule covalently bound to protein via a cysteine linkage at the number 3 carbon of APAP. Embodiments of the anti-APAP antibody 31 and methods of synthesizing the anti-APAP antibody 31 are described in Roberts et al. 1987, Potter et al. 1989, and Mathews et al. 1996, all of which are incorporated by reference in their entirety herein. The anti-APAP antibody 31 may be monoclonal, polyclonal, chimeric, humanized, and combinations thereof. In other embodiments, the anti-APAP antibody 31 may recognize any aspect of the APAP molecule presented as an antigenic determinant by virtue of covalent binding of the reactive metabolite NAPQI to a cysteine residue of the peptide or protein forming an APAP-protein adduct or APAP-protein complex.

### VI. Anti-APAP Antibody Coupled to Indicator

A known anti-APAP antibody 31 may be coupled to a known indicator compound to form an anti-APAP antibody coupled to indicator 32. In one embodiment, the anti-APAP antibody coupled to indicator 32 causes an indicator change in the substrate 30 whenever there exists a suitably dense concentration of the indicator localized in the substrate 30. In an embodiment, the indicator change may occur at the test zone 40 of the substrate 30. The conditions under which an indicator change may occur in the device 10 vary depending on the specific embodiment of the device 10.

### VII. Synthetic APAP-Protein Adducts

Synthetic APAP-protein adduct 34 may bind to the anti-APAP antibody 31 in competition with any APAP-protein adduct 22 present in the sample 20. The synthetic APAP-protein adduct 34, like the APAP-protein adduct 22 in the sample 20, is an APAP molecule bound to a cysteine residue of a protein. Alternatively, synthetic APAP-protein adduct 34 may be APAP coupled to proteins or peptides, using other linkages that present APAP as a bound antigenic hapten. Exemplary synthetic APAP-protein adduct 34 includes APAP bound to proteins or peptides containing available free cysteinyl sulfhydryl groups, including but not limited to bovine serum albumin (BSA), ovalbumin, lactalbumin, peptides bearing cysteine residues, and combinations thereof.

### VIII. Synthetic APAP-Protein Adducts Coupled to Indicator

The synthetic APAP-protein adduct 34 described above may be coupled to an indicator, forming a synthetic APAP-protein adduct coupled with indicator 35. Synthetic APAP-protein adduct coupled to indicator 35 may cause an indicator change in the substrate 30 whenever there exists a suitably dense concentration of the indicator localized in the substrate 30. In an embodiment, the indicator change may occur at the test zone 40 of the substrate 30. The conditions under which an indicator change may occur in the device 10 vary depending on the specific embodiment of the device 10. Depending on the configuration of the device 10, the indicator change may be either be proportional or inversely proportional to the concentration of APAP-protein adduct 22 in the sample.

### IX. Indicators

In one embodiment, an indicator is used to cause a measurable change in a region of the device 10 in response to the presence or absence of APAP-protein adduct 22 or synthetic APAP-protein adduct 34 in the sample 20. Depending on the embodiment of the device 10, the indicator may be coupled to an anti-APAP antibody 31, a synthetic APAP-protein adduct 34, or a control compound coupled to indicator 36. The indicator coupled to the control compound may be the same or different from the indicator coupled to the anti-APAP-protein adduct antibody or coupled to synthetic APAP-protein adduct. In another embodiment, the indicators may cause a detectible change in a region of the substrate 30 when the indicator is densely concentrated in a region of the substrate 30. In yet another embodiment, the indicators may cause a detectible change in a region of the substrate 30 when the indicator reacts with a reagent that is adhered to the substrate 30 in a region such as a test zone 40 or a control zone 42 of the substrate 30.

### A. Visual Indicators

A visual indicator may register a change by absorbing specific wavelengths of light resulting in the reflection of a limited subset of the wavelengths of light illuminating the substrate 30, by fluorescing light after being illuminated, or by emitting light via chemiluminescence. The indicator change registered by the indicators may be in the visible light spectrum, the infrared spectrum, or the ultraviolet spectrum. Non-limiting examples of visual indicators suitable for the device 10 include nanoparticulate gold, organic particles such as polyurethane or latex microspheres loaded with dye compounds, carbon black, fluorophores, radioactive isotopes, nanoparticles, enzymes such as horseradish peroxidase or alkaline phosphatase that react with a chemical substrate to form a colored product, and combinations thereof.

### B. Electrochemical Indicators

An electrochemical indicator may register a indicator change by altering an electrical property of the substrate. The change registered by the indicators may be an alteration in the conductivity of the substrate, the resistance of the substrate, the capacitance of the substrate, the current conducted by the substrate in response to an applied voltage, the voltage required to achieve a desired current through the substrate, and combinations thereof. In one embodiment, the electrochemical indicators may include redox species including ascorbate (vitamin C), vitamin E, glutathione, polyphenols, catechols, quercetin, phytoestrogens, penicillin, carbazole, murranes, phenols, carbonyls, benzoates, trace metal ions such as nickel, copper, cadmium, iron and mercury, and combinations thereof.

### X. Control Compound Coupled to Indicator and Control Capture Agent

The control compound 33 is a molecule that diffuses through the substrate 30 at a rate similar to that of the APAP-protein adduct 22, the synthetic APAP-protein adduct coupled to indicator 35 and the anti-APAP antibody coupled to indicator 32. In addition, the control compound 33 is a molecule that does not react or otherwise interfere with the transport or conjugation of the other reagents in the substrate 30. In one embodiment, the control compound 33 is coupled to an indicator to form a control compound coupled to indicator 36.

The control compound coupled to indicator 36, in a manner similar to the anti-APAP antibody 31 in the substrate 30, is dissolved in the solvent of the sample 20 and is transported along the substrate 30 by a wicking action. However, the control compound coupled to indicator 36 binds with the control capture agent 38 that is adhered to the substrate 30 at the control zone 42. The resulting indicator change at the control zone 42 indicates that the sample 20 was properly absorbed and transported down the length of the substrate 30 of the device 10.

Suitable control compounds 33 may also include a reactive compound such as an enzyme that reacts with the control capture agent 38 to form an indicator change. Indicators suitable for coupling to the control compound 33 are described above.

The control capture agent 38 is a compound that is capable of adhering to the porous substrate 30, as well as preferentially binding to the control compound 33. In an exemplary embodiment, the control compound coupled to indicator 36 is streptaviden coupled to nanoparticulate gold and the control capture agent 38 is biotinylated bovine serum albumin.

### XI. Lateral Flow Immunochromatographic Assay Device

Lateral flow immunochromatographic assay devices 10 are exemplary embodiments of the device 10 described above, one embodiment of which is shown pictured in FIG. 1. The lateral flow immunochromatographic assay device 10 includes a porous substrate 30, an amount of anti-APAP antibody coupled to indicator 32, and an amount of a synthetic APAP-protein adduct 34. In addition, the lateral flow immunochromatographic assay device 10 may also include a control compound coupled to indicator 36, and a control capture agent 38.

Referring to FIG. 1, in one embodiment, the anti-APAP antibody coupled to indicator 32 and the control compound coupled to indicator 36 are diffusively attached to the substrate 30 at the sample contact end 45. The synthetic APAP-protein adduct 34 is adhered to the substrate 30 in a test zone 40. The control capture agent 38 is adhered to the substrate 30 as well in a control zone 42. The synthetic APAP-protein adduct 34 is adhered to the substrate 30 at the test zone 40 and this is the location of any indicator changes due to the absence of APAP-protein adduct 22 in the sample 20. Indicator changes to indicate the proper function of the lateral flow immunochromatographic assay device 10 occur at the control zone 42.

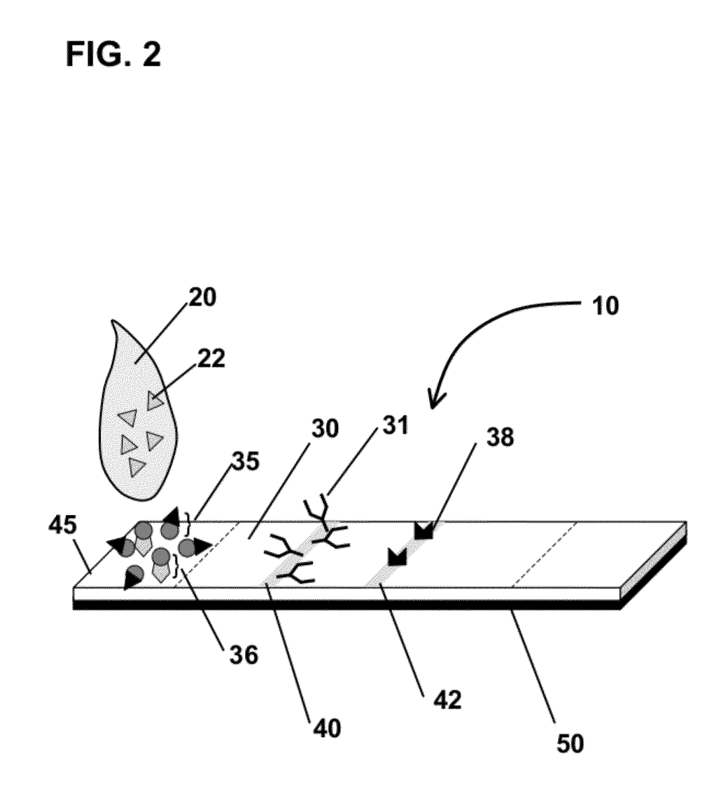

In another embodiment of a lateral flow immunochromatographic assay device 10, shown in FIG. 2, the synthetic APAP-protein adduct coupled to an indicator 35 and the control compound coupled to indicator 36 are diffusively attached to the substrate 30 at the sample contact end 45. In this embodiment, the anti-APAP antibody 31 may be adhered to the substrate 30 in a test zone 40. The control capture agent 38 is adhered to the substrate 30 in a control zone 42. The anti-APAP antibody 31 is adhered to the substrate 30 at the test zone 40 and this is the location of any indicator changes due to the absence of APAP-protein adduct 22 in the sample 20. Indicator changes to indicate the proper function of the lateral flow immunochromatographic assay device 10 occur at the control zone 42.

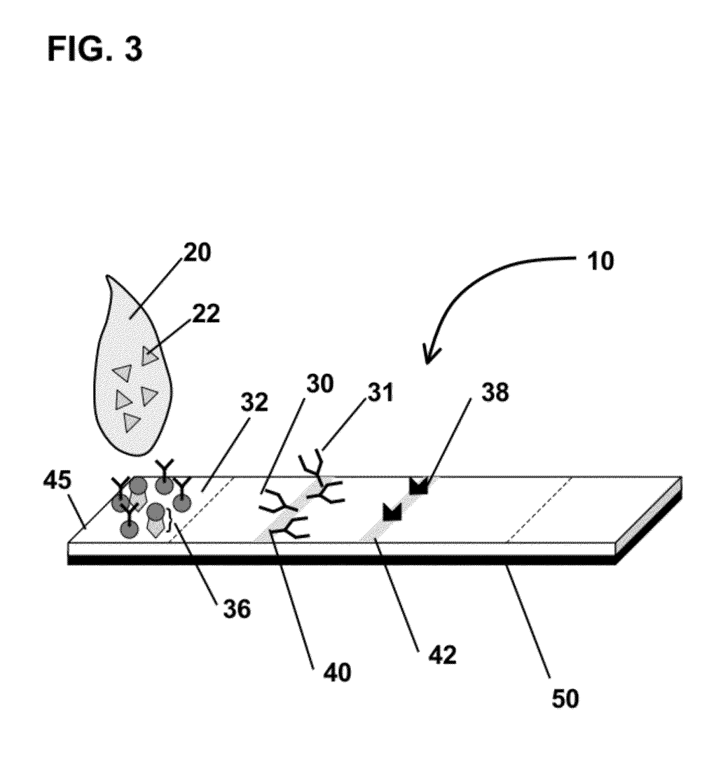

Referring to FIG. 3, yet another embodiment of a lateral flow immunochromatographic device 10 includes an anti-APAP antibody coupled to indicator 32 that is diffusively attached to the substrate 30 at the sample contact end 45. In this embodiment, a second anti-APAP antibody 31 is adhered to the substrate 30 in a test zone 40. Indicator changes due to the presence of APAP-protein adduct 22 in the sample 20 occur at the test zone 40, as described above. In addition, control compound coupled to indicator 36 and control capture agent 38 may be included in this embodiment in a manner similar to the embodiments described above.

### XII. Assay Approaches

### A. Competitive Assay

Various embodiments of the chromatographic device 10 utilize a competitive assay approach to detect APAP-protein adduct 22. In these embodiments, shown in FIG. 1, the sample 20 is contacted with the sample contact end 45 of the substrate 30. The solvent of the sample 20 dissolves the anti-APAP antibody coupled to indicator 32 and control compound coupled to indicator 36 that were diffusively attached to the porous substrate 30 near the contact point of the sample 20, as shown in FIG. 3.

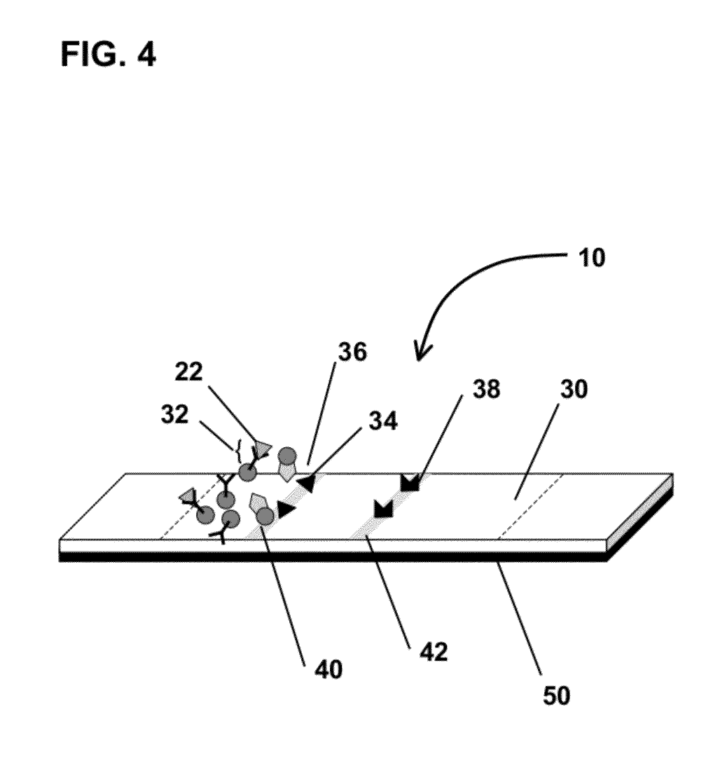

FIG. 4 depicts the progression of the assay as the solvent wicks toward the opposite end of the substrate 30. Any available APAP-protein adduct 22 in the sample 20 binds to the anti-APAP antibody coupled to indicator 32. The movement of the solvent of the sample 20 through the substrate 30 transports the dissolved reagents through the substrate 30 as well.

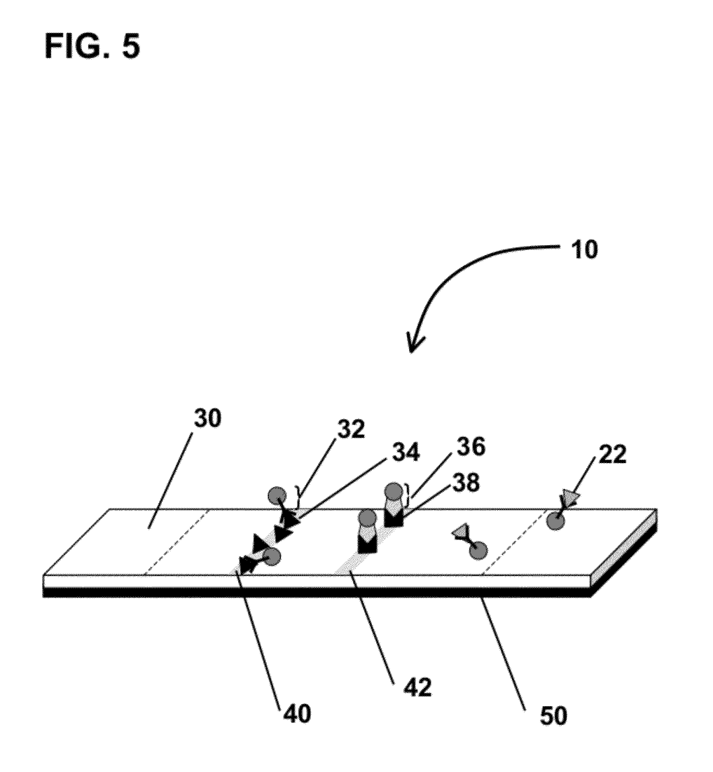

When the solvent of the sample 20 encounters the synthetic APAP-protein adduct 34 that is adhered to the substrate 30 at the test zone 40, as shown in FIG. 5, any remaining anti-APAP antibody bound to indicator 32 that was not bound to the APAP-protein adduct 22 of the sample 20 bind to the synthetic APAP-protein adduct 34, and are immobilized at the test zone 40. If little or no APAP-protein adduct 22 is present in the sample 20, a high concentration of anti-APAP antibody coupled to indicator 32 will occur at the test zone 40, resulting in an indicator change.

When the solvent of the sample 20 encounters the immobilized control capture agents 38 at the control zone 42, also shown in FIG. 5, the control capture agent 38 binds to the control compound coupled to indicator 36, resulting in an indicator change at the control zone 42 due to the high density of control compound coupled to indicator 36 in the control zone 42. An indicator change at the control zone 42 may be used as an indication that the device 10 is functioning properly.

In summary, if the sample 20 tested by the dipstick device 10 with a competitive assay embodiment as described above contains APAP-protein adduct 22, then the majority of the anti-APAP antibody 31 will be bound to APAP-protein adduct 22, resulting in no indicator change at the test zone 40. If the sample 20 tested by the dipstick device 10 as described above does not contain APAP-protein adduct 22, then the majority of the anti-APAP antibody 31 will not bind with APAP-protein adduct 22, resulting in an indicator change at the test zone 40. The intensity of the indicator change at the test zone 40 is an inverse function of the amount of APAP-protein adduct 22 in the sample 20, and may additionally be quantified using densitometry or other means known in the art.

In an alternative embodiment, such as the device 10 shown in FIG. 2, anti-APAP antibody 31 is adhered to the substrate 30 at the test zone 40. After the sample 20 is contacted with the sample contact end 45 of the substrate 30 the solvent of the sample 20 wicks toward the test zone 40 carrying dissolved synthetic APAP-protein adduct coupled to indicator 35 along with the sample 20. Synthetic APAP-protein adduct coupled to indicator 35 compete with any APAP-protein adduct 22 in the sample 20 for binding the limited amount of anti-APAP antibody 31 immobilized at the test zone 40. In this embodiment, the accumulation of the indicator (and associated indicator change) is inversely related to the concentration of APAP-protein adduct 22 in the sample 20.

### B. Non-Competitive Assay

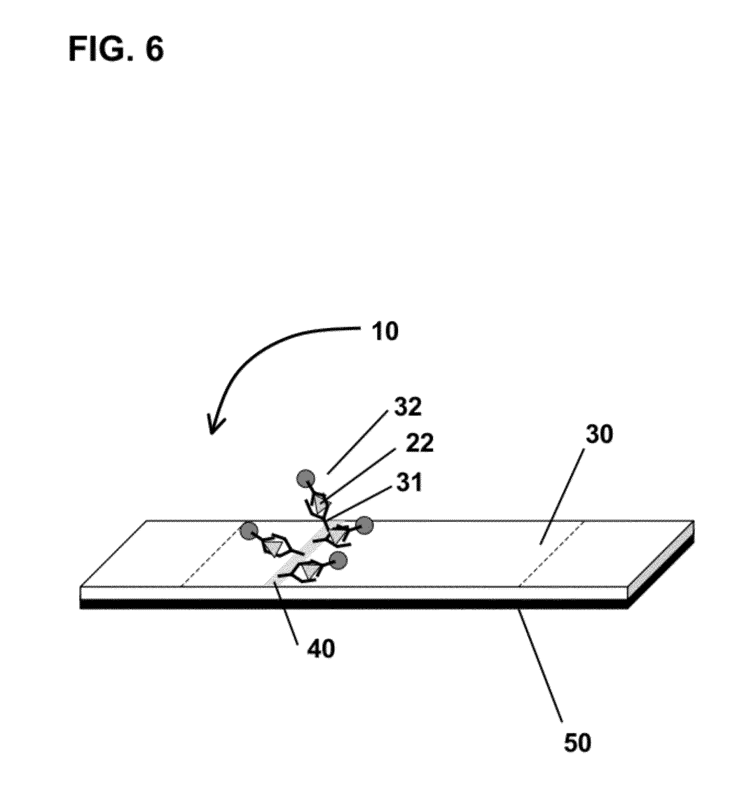

Embodiments of the device 10 may utilize a non-competitive assay approach to detect APAP-protein adduct 22. In one embodiment, shown in FIG. 3, the sample 20 is contacted with the substrate 30 at the sample contact end 45. The solvent of the sample 20 dissolves the anti-APAP antibody coupled to indicator 32 and wicks the anti-APAP antibody coupled to indicator 32 as well as any APAP-protein adduct 22 in the sample 20 toward the test zone 40. As the solvent wicks toward the distal end 47 of the substrate 30, any available APAP-protein adduct 22 binds with the anti-APAP antibody coupled to indicator 32. A second group of anti-APAP antibody 31 is adhered to the substrate 30 at the test zone 40 and function as capture antibodies. When the solvent of the sample 20 encounters the immobilized anti-APAP antibody 31, the APAP-protein adduct 22 binds to the immobilized anti-APAP antibody 31, as shown in FIG. 6. If APAP-protein adduct 22 is present in the sample 20, a high concentration of anti-APAP antibody coupled to indicator 32, which are also bound to the APAP-protein adduct 22, will occur at the test zone 40, resulting in an indicator change.

In an alternative embodiment, the immobilized antibody 31 at the test zone 40 may be an antibody with binding specificity for one or more specific proteins that form APAP-protein adducts. If the protein captured by the immobilized antibody 31 is part of an APAP-protein adduct, then density of anti-APAP antibody coupled to indicator 32 will increase at the test zone 40, causing an indicator change.

### C. Quantitative Assays

In embodiments of the devices 10 described above, the concentrations of reagents contained in the substrate 30 may be optimized to yield indicator changes when the APAP-protein adduct 22 concentration in the sample 20 falls within a certain range. In this manner, certain embodiments of the devices 10 may be made quantitative. In other embodiments, the indicator changes in the substrate of the devices 10 may be detected by a densitometer or other means known in the art, yielding quantitative measurements of serum APAP-protein adduct 22 concentrations.

### XIII. Method of Measuring APAP-Protein Adduct in a Sample

Another embodiment provides a method of determining an amount of APAP-protein adduct 22 in a sample 20. The method includes contacting an amount of the sample 20 with a substrate 30 containing an amount of anti-APAP antibody coupled to indicator 32 and a synthetic APAP-protein adduct 34. The amount of APAP-protein adduct 22 in the sample 20 is then determined by measuring the indicator change caused by the binding of the anti-APAP antibody coupled to indicator 32 to the synthetic APAP-protein adduct 34.

In several iterations of the embodiments, the concentrations of the anti-APAP antibody and the synthetic APAP-protein adduct 34 or the pore size of the substrate and associated rate of sample wicking may be manipulated to adjust the sensitivity of the device 10. In one embodiment, the device 10 is sensitive to changes in serum APAP-protein adduct 22 concentration ranging between about 0.1 nmol/ml of serum and about 100 nmol/ml of serum. In other embodiments, the device 10 is sensitive to changes in serum APAP-protein adduct 22 concentration ranging between about 0.5 nmol/ml of serum and about 10 nmol/ml of serum, between about 0.5 nmol/ml of serum and about 80 nmol/ml of serum, between about 1 nmol/ml of serum and about 60 nmol/ml of serum, between about 1 nmol/ml of serum and about 50 nmol/ml of serum, between about 1 nmol/ml of serum and about 40 nmol/ml of serum, between about 1 nmol/ml of serum and about 30 nmol/ml of serum, between about 1 nmol/ml of serum and about 20 nmol/ml of serum, and between about 1 nmol/ml of serum and about 10 nmol/ml of serum. In an exemplary embodiment, the device 10 is sensitive to changes in serum APAP-protein adduct 22 concentrations ranging between about 1 nmol/ml of serum and about 40 nmol/ml of serum.

### DEFINITIONS

To facilitate understanding of the invention, a number of terms and abbreviations as used herein are defined below:

The term “competitive assay” generally refers to an immunological assay method in which the target analyte and a synthetic version of the analyte compete to bind with the antibody of the assay. The antibody may be in solution, or the antibody may be immobilized, depending on the specific assay embodiment.

The term “non-competitive assay” generally refers to an immunological assay method, also known as a sandwich or capture assay, in which the target analyte or a compound or structure containing the analyte binds with an immobilized capture antibody, as well as with a second antibody that is coupled to an indicator. The capture antibody may have specificity for either the analyte, or the compound or structure containing the analyte.

The term “diffusively attached” generally refers to the manner in which mobile reagents are present in the substrate of a lateral flow immunochromatographic device. The reagents may be contacted with the substrate in solution and then dried, leaving the reagents behind on the substrate but not attached to the substrate. When a sample is contacted with the substrate, the reagents are dissolved by the solvent of the sample, and are transported diffusively along the substrate.

### EXAMPLES

The following examples illustrate several aspects of the invention.

### Example 1

### APAP-Protein Adduct was Measured in Clinical Samples Using the HPLC-EC Assay in Adults with Acute Liver Failure

To demonstrate the feasibility of detecting acetaminophen (APAP) overdoses using an assay for the APAP-protein adduct the following study was conducted. APAP-protein adducts were measured in the serum samples of 53 patients hospitalized after suicidal APAP overdoses that resulted in acute liver failure (ALF). Serum samples were obtained daily from patients over a seven-day hospital stay. Sixty-eight percent of the patients were females, and the mean age of the patients was 33.6±12.1 yrs (mean±SD). The patients of this study had ingested known large amounts of APAP (468±284 mg/kg) an average of 74.8±33.4 hrs prior to hospital admission. Nine of the 53 patients died.

Serum samples were analyzed for acetaminophen-protein adducts (as measured by acetaminophen-cysteine or APAP-CYS) using a high-performance liquid chromatography with electrochemical detection (HPLC-EC) method. All serum samples were dialyzed, treated with protease, and then precipitated with trichloracetic acid. Following centrifugation, APAP-CYS in the resulting supernate was quantified as a measure of APAP-protein adducts using HPLC-EC (Model 582 solvent delivery system with a Model 5600A CoulArray detector; ESA, Chelmsford, Mass.).

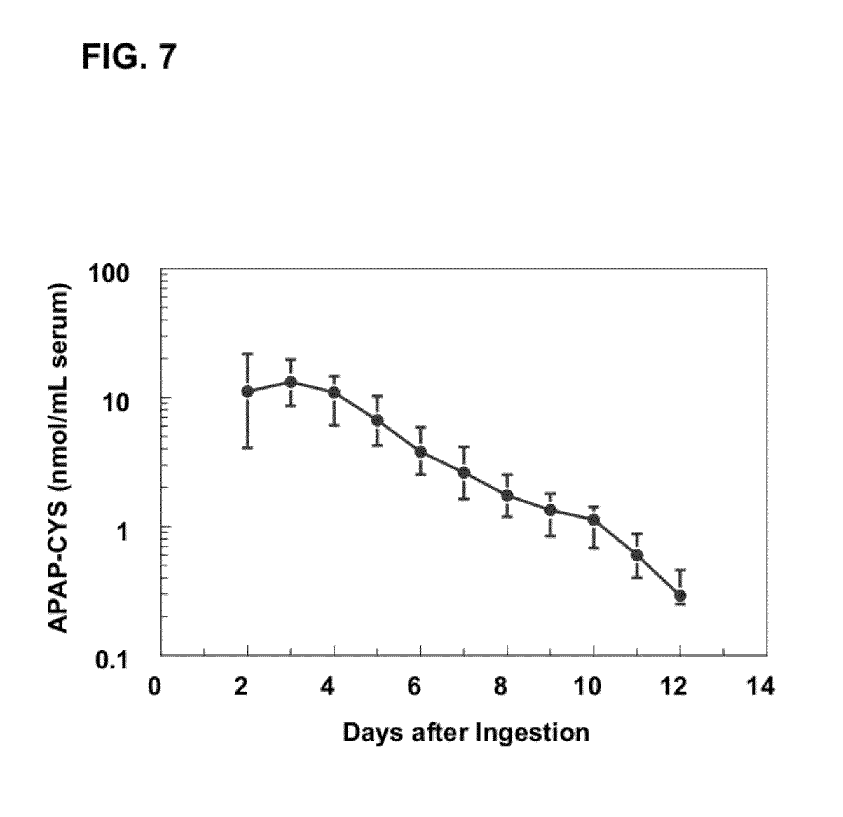

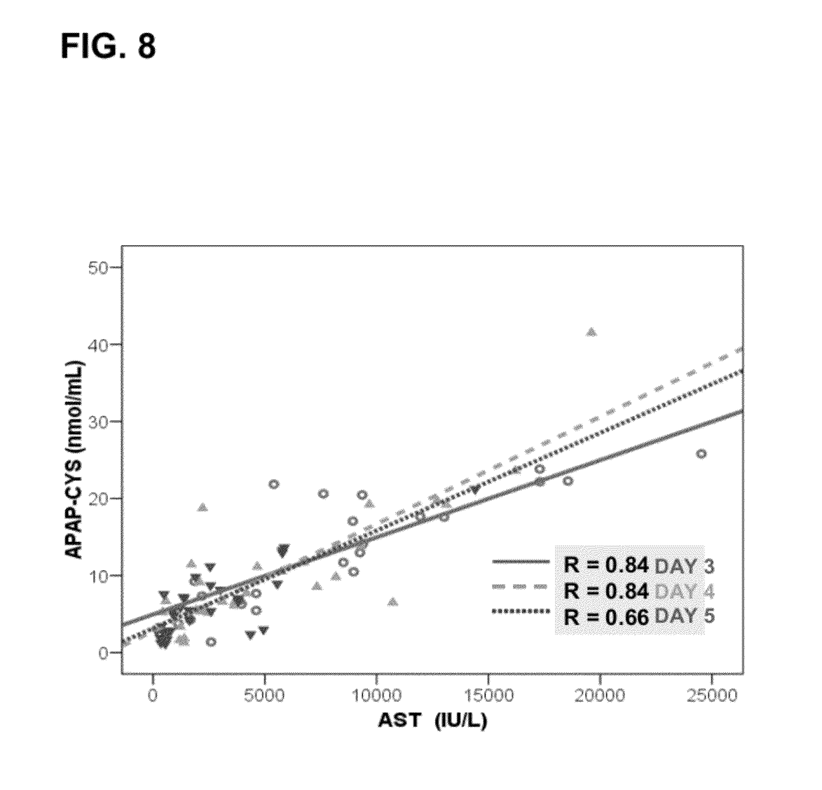

FIG. 7 shows the mean APAP-protein adduct concentrations measured in the study serum samples as a function of time elapsed after the initial APAP overdose. The measured APAP-protein adduct levels were elevated in the first samples obtained in the study and persisted above the threshold level for APAP toxicity for twelve days after the initial overdose. FIG. 8 shows correlations between serum APAP-protein adduct concentration and serum aspartate aminotransferase (AST) concentration, a non-specific biomarker used to diagnose liver toxicity. The correlations were derived using serum APAP-protein adduct concentrations and peak serum AST concentrations measured 3, 4, and 5 days after the initial APAP overdose.

In addition, a pharmacokinetic analysis was performed to characterize the elimination of APAP-protein adducts from the patients of this study. Individual empiric Bayesian estimates were determined for each of 20 patients with 4 consecutive daily serum samples available analysis. The mean kₑ (elimination rate constant) was 0.402±0.05 day⁻¹. The mean elimination half-life was 1.75±0.21 days.

The results of this study indicated that measured serum APAP-protein adducts were a valid and specific bioindicator of APAP overdose. Adducts remained in blood 12 days after the APAP overdose. This diagnostic window far exceeded the diagnostic detection period for the parent compound, APAP, which has a reported elimination half-life of about 18 hours under overdose conditions. Thus, the window for diagnosis of APAP-overdose by detection of APAP-protein adducts far exceeded the window for diagnosis of APAP-overdose based on detection of the parent compound, which is the basis of the Rumack nomogram.

### Example 2

### APAP-Protein Adduct was Measured in Clinical Samples Using the HPLC-EC Assay in Adolescents and Children with APAP Overdose

To demonstrate the feasibility of detecting acetaminophen (APAP) overdoses in a pediatric population, the following study was conducted. APAP-protein adducts and AST concentrations were measured in the serum samples of 157 adolescents and children that were victims of APAP overdose using the methods described in Example 1. All patients had full recovery and one patient required a liver transplant. The severity of liver injury was stratified by the highest recorded value for AST concentration.

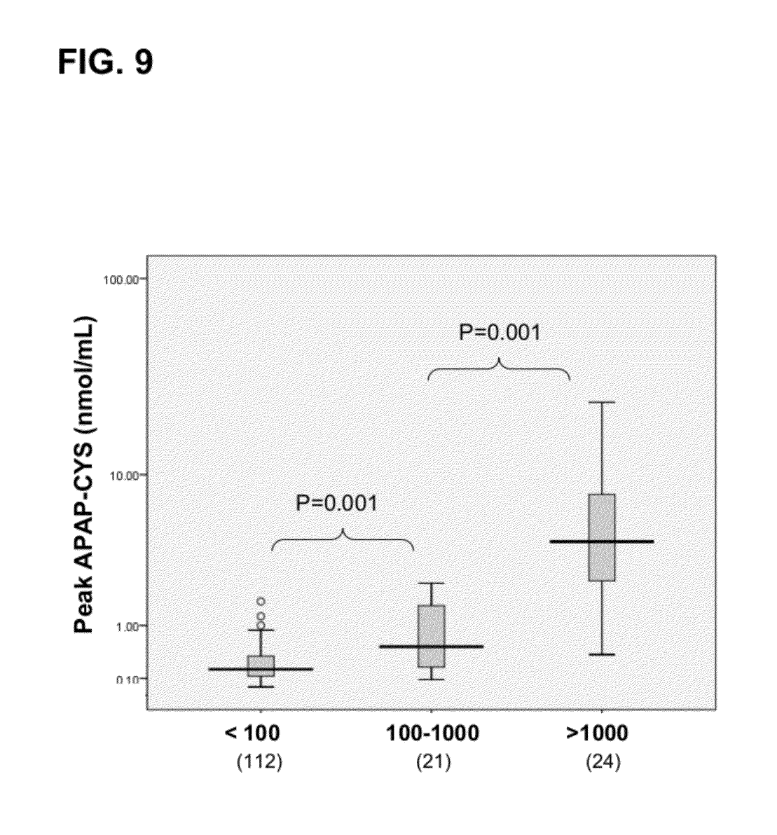

FIG. 9 shows box plots of the median/range values for APAP-protein adducts for three different subgroups of children and adolescents with APAP overdose. The subgroups were based on the severity of toxicity defined as the highest recorded value for AST concentration for each patient over the period of hospitalization. The boxes in FIG. 9 represent the 25th-75th interquartile ranges for the subgroups and the horizontal bars in the boxes represent the median values for each subgroup. Elevated levels of the specific biomarker, APAP-protein adducts, correlated with elevated levels of the traditional, but nonspecific, correlate of liver toxicity, AST. Significant differences in APAP-protein adduct levels were detected between the toxicity severity subgroups. In addition, higher values of adducts were detected in patients that had delays in treatment with the antidote for APAP toxicity, N-acetylcysteine. Adduct concentrations were also associated with risk predictions for the development of toxicity, based on the currently used clinical tool for risk stratification (Rumack nomogram). In a pharmacokinetic analysis of this data, it was determined that the mean (±SD) kₑ and half-life of adducts in serum in this population were 0.486±0.084 days⁻¹ and 1.47±0.30 days, respectively. Adducts were present in blood 9 days after the overdose.

The results of this study indicated that the measured serum levels of APAP-protein adducts were useful bioindicators for APAP overdose. In addition, adducts persisted in the serum much longer than the parent compound, APAP. Further, the results of this study indicated that the measured serum levels of APAP-protein adduct could be used to determine the severity of the overdose in a population of children and adolescent patients.

### REFERENCES

Potter D W, Pumford N R, Hinson J A, Benson R W, Roberts D W 1989. “Epitope characterization of acetaminophen bound to protein and nonprotein sulfhydryl groups by an enzyme linked immunosorbent assay”. J Pharmacol Exp Ther. 248:182-189.
Mathews A. M., Roberts D. W., Hinson J. A., Pumford N. R. 1996. “Acetaminophen-induced hepatotoxicity: Analysis of total covalent binding vs. specific binding to cysteine”. Drug Metab Dispos. 24:1192-1196.
Roberts D. W., Pumford N. R., Potter D. W., Benson R. W., Hinson J. A. 1987. “A sensitive immunochemical assay for acetaminophen-protein adducts”. J. Pharmacol Exp. Ther. 241:527-533.

What is claimed is:

1. A method of determining an amount of APAP-protein adduct in a sample, comprising:
a. contacting an amount of the sample with a substrate comprising an anti-APAP antibody coupled to an indicator and further comprising a synthetic APAP-protein adduct; and,
b. determining the amount of APAP-protein adduct in the sample by measuring an indicator change caused by inhibition of binding of the synthetic APAP-protein adduct to the anti-APAP antibody coupled to the indicator.

2. The method of claim 1, wherein the amount of APAP-protein adduct detectible in the sample ranges between about 0.1 nmol/ml of serum and about 100 nmol/ml of serum.

3. The method of claim 2, wherein the amount of APAP-protein adduct detectible in the sample ranges between about 1 nmol/ml of serum and about 40 nmol/ml of serum.

4. The method of claim 1, wherein the sample is selected from the group consisting of blood, urine, saliva, tears, breast milk, lymph, blood plasma, blood serum, bile fluid, cerebrospinal fluid, supernate from cell cultures, tissue extracts, and combinations thereof.

5. The method of claim 1, wherein the sample is conditioned prior to contact with the substrate using methods selected from the group consisting of centrifugation, protein precipitation, fast gel filtration with a molecular weight cutoff of about 5 kDa, and combinations thereof.

6. The method of claim 1, wherein the anti-APAP antibody coupled to an indicator is transported to the synthetic APAP-protein adduct by wicking through the substrate.

7. The method of claim 1, wherein the indicator change is measured using an instrument selected from the group consisting of densitometer, fluorometer, quantitative voltammetry device, and quantitative coulometry device.

8. A method of determining an amount of APAP-protein adduct in a sample, comprising:
a. contacting an amount of the sample with a substrate comprising an amount of a synthetic APAP-protein adduct coupled to an indicator and further comprising an anti-APAP antibody; and,
b. determining an amount of APAP-protein adduct in a sample by measuring an indicator change caused by inhibition of binding of the anti-APAP antibody to the synthetic APAP-protein adduct coupled to an indicator.

9. The method of claim 8, wherein the amount of APAP-protein adduct detectable in the sample ranges between about 0.1 nmol/ml of serum and about 100 nmol/ml of serum.

10. The method of claim 9, wherein the amount of APAP-protein adduct detectable in the sample ranges between about 1 nmol/ml of serum and about 40 nmol/ml of serum.

11. The method of claim 8, wherein the sample is selected from the group consisting of blood, urine, saliva, tears, breast milk, lymph, blood plasma, blood serum, bile fluid, cerebrospinal fluid, supernate from cell cultures, tissue extracts, and combinations thereof.

12. The method of claim 8, wherein the sample is conditioned prior to contact with the conjugated antibody using methods selected from the group consisting of centrifugation, protein precipitation, fast gel filtration with a molecular weight cutoff of about 5 kDa, and combinations thereof.

13. The method of claim 8, wherein the synthetic APAP-protein adduct is transported to the anti-APAP antibody by wicking through the substrate.

14. The method of claim 8, wherein the indicator change is measured using an instrument selected from the group consisting of densitometer, fluorometer, quantitative voltammetry device, and quantitative coulometry device.

15. A method of determining an amount of an APAP-protein adduct in a sample, comprising:
a. contacting an amount of the sample with a substrate comprising an amount of a first anti-APAP antibody conjugated with an indicator and further comprising a second anti-APAP antibody; and,
b. determining the amount of APAP-protein adduct in the sample by measuring indicator changes caused by the binding of the APAP-protein adduct to the first anti-APAP antibody and to the second anti-APAP antibody.

16. The method of claim 15, wherein the amount of APAP-protein adduct detectable in the sample ranges between about 0.1 nmol/ml of serum and about 100 nmol/ml of serum.

17. The method of claim 16, wherein the amount of APAP-protein adduct detectable in the sample ranges between about 1 nmol/ml of serum and about 40 nmol/ml of serum.

18. The method of claim 15, wherein the sample is selected from the group consisting of blood, urine, saliva, tears, breast milk, lymph, blood plasma, blood serum, bile fluid, cerebrospinal fluid, supernate from cell cultures, tissue extracts, and combinations thereof.

19. The method of claim 15, wherein the sample is conditioned prior to contact with the conjugated antibody using methods selected from the group consisting of centrifugation, protein precipitation, fast gel filtration with a molecular weight cutoff of about 5 kDa, and combinations thereof.

20. The method of claim 15, wherein the APAP-protein adduct is transported to the first and second anti-APAP antibody by wicking through the substrate.
